(19) United States
(12) Patent Application Publication
(10) Pub. No.: US 2004/0048819 A1
(43) Pub. Date: Mar. 11, 2004

(54) Complexes for transferring nucleic acids into cells

(76) Inventors: Joachim Simon, Dusseldorf (DE); Martin Vollmer, Leverkusen (DE); Ulrich Betz, Wuppertal (DE); Philip Scuderi, Chapel Hill, NC (US)

Correspondence Address: BAYER POLYMERS LLC, 100 BAYER ROAD, PITTSBURGH, PA, 15205, US

(21) Appl. No.: 10/398,561

(22) PCT Filed: Oct. 1, 2001
(86) PCT No.: PCT/EP01/11317

(30) Foreign Application Priority Data
Oct. 9, 2000 (DE) 100 49 808.6
Oct. 23, 2000 (DE) 100 52 479.6
Sep. 12, 2001 (DE) 101 45 134.2

(51) Int. Cl. A61K 48/00

(52) U.S. Cl. 514/44

(57) ABSTRACT

The invention relates to complexes of cationic polymers and nucleic acids, to the use of such complexes for introducing nucleic acids into cells and organisms, to the use of the complexes as pharmaceuticals, and to novel polymers which can be used to prepare the complexes. The polymers are preferably polyamines and more preferably polyethyleneimine.

The invention relates to complexes of cationic polymers and nucleic acids, to the use of such complexes for introducing nucleic acids into cells, and to the use of the complexes as pharmaceuticals. The invention also relates to novel polymers which can be used to prepare the complexes.

It has not to date been possible to achieve continuing success in the therapeutic use of nucleic acids (DNA and RNA) in vivo in humans. The reasons for this are presumably the limited expression of the necessary genetic information, which is in turn caused by an inadequate efficiency of gene transfer or of the availability of the nucleic acids to be expressed. Additional reasons playing an important part are the inadequate stability of the transport or vector systems used, and inadequate biocompatibility.

The possibility of oral or intranasal administration of nucleic acids for gene therapy or immunization is particularly attractive (Page & Cudmore, Drug Discovery Today 2001, 6, 92-101). In this case it is essential to protect the nucleic acids from breakdown by nucleases. In the case of vaccination in particular exposure of the mucous membranes is preferable to parenteral administration in order to ensure stimulation of MALT (mucosa associated lymphoid tissue), which is involved in the immunological protection of the mucous membranes. Prevention of infections in this region is of great importance for example with pathogens such as HIV (human immunodeficiency virus) or HSV (herpes simplex virus).

Viral vectors such as retroviruses or adenoviruses entail the risk of inducing inflammatory or immunogenic processes (Mc Coy et al., Human Gene Therapy 1995, 6, 1553-1560; Yang et al., Immunity 1996, 1, 433-442).

There has been work done on nonviral, synthetic transport systems as alternatives, but they do not yet show the desired properties. Systems based in particular on mixtures of lipids and, where appropriate, other admixed cell-specific ligands can be characterized biophysically only with difficulty or inadequately and moreover entail the risk of dynamic structure-changing processes on storage and administration. In particular, safety of administration as a precondition for use as pharmaceuticals is not present in this case.

Complexes based on synthetic cationic polymers are therefore preferred as long as their structural features can be prepared reproducibly and be unambiguously characterized (M. C. Garnett, Critical Reviews in Therapeutic Drug Carrier Systems 1999, 16, 147-207).

Numerous processes described for preparing synthetic cationic polymers for preparing complexes lead to undefined products with regard to the degree of branching of the polymers and their microstructure. In addition, numerous polymers employed for transfection are characterized only by very broad molecular weight distributions or described only by their average molecular weights.

Polyethyleneimine (PEI), a cationic polymer with a three-dimensional, branched structure, is particularly suitable for complexation and condensation of nucleic acids (W. T. Godbey, J. of Controlled Release 1999, 60, 149-160). It was possible in a number of in vitro experimental series to show the suitability for introducing nucleic acids into cells, and polymers with low molecular weights (LMW-PEI, LMW: low molecular weight) in the region of MW 2000 g/mol in particular showed high activity (EP-A 0 905 254). The undefined structure of the branched polymers is to be regarded as a disadvantage thereof.

Linear polyethyleneimines by contrast can be prepared with defined molecular weights and have been employed in numerous applications for in vitro and in vivo gene transfer (WO 96/02655). Efforts to improve the transfection efficiency of the linear polyethyleneimines has led in two directions (M. C. Garnett, Critical Reviews in Therapeutic Drug Carrier Systems 1999, 16, 147-207):

1) Through introducing hydrophilic substituents on the one hand it was possible to increase the solubility of the DNA/polymer complexes in water, and on the other hand it was possible to make the complexes inert with regard to interaction with proteins. In addition, block copolymers of polyethylene glycol and polyethyleneimine have also been described.

2) It was possible to achieve a targeting effect by introducing cell-specific ligands, usually hydrophilic carbohydrate or peptide structures.

The efficiency of transfer of the complexed nucleic acids into cells depends on many factors, especially on the interaction between complexes and cell membranes, the nature of the cell type, the size of the complexes and the charge ratio between the components of the complex. Little is known about the interaction between complexes and cell membrane, and about uptake in cells.

It was possible to show an increased interaction between polyethyleneimines with hydrophobic substituents and model membranes consisting of anionic phospolipids on the basis of a comparison of branched unsubstituted polyethyleneimines with substituted polyethyleneimines by a degree of substitution with hexyl or dodecyl alkyl chains of up to 50 mol % (D. A. Tirell et al., Macromolecules 1985, 18, 338-342).

The use of polyethyleneimines with hydrophobic functionalities for complexation of nucleic acids has been described only for alkyl-substituted systems (WO 99/43752). It was additionally possible to show for cationic polymers based on polyacrylates that hydrophobic monomer units increase the transfection efficiency (M. Kurisawa et al., J. Controlled Release 2000, 68, 1-8). It was possible to show for hydrophobicized poly-L-lysine with 25 mol % stearyl units that ternary complexes of nucleic acids with lipoproteins in combination with these polymers lead to an increase in the transfection efficiency in muscle cells (K.-S. Kim, J. of Controlled Release 1997, 47, 51-59). EP-A 0 987 029 describes polyallylamines which may optionally have linear and branched alkyl chains or else aryl groups.

Hydrophobized polyethyleneimines with long-chain alkyl radicals have already been employed in the form of quaternary, completely alkylated and thus highly charged structures as catalyst systems in, for example, ester cleavages. In addition, acylated structures have also been employed for stabilizing enzymes (U.S. Pat. No. 4,950,596).

The present invention relates to complexes which comprise a linear cationic polymer which is soluble or dispersible in water and has hydrophobic substituents, and at least one nucleic acid.

The polymer is preferably a polyamine and particularly preferably a polyethyleneimine.

The hydrophobic substituents can be disposed as side chains or terminally on the polymer. The degree of substitution (percentage content of functionalized N atoms in the main polymer chain) is preferably between 0.01 and 10 percent.

Particularly suitable hydrophobic substituents are alkyl chains, acyl chains or steroid-like substituents. Acyl chains are especially suitable as hydrophobic substituents. Also suitable are hydrophobic substituents which can be introduced by addition of the nitrogen functions of the main polymer chain onto isocyanates or onto α,β-unsaturated carbonyl compounds.

A polymer which can preferably be used for the complex formation has the following general formula:

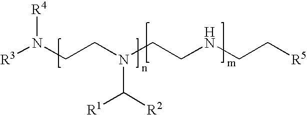

in which in each individual [CH₂—CH₂—N] unit PS

R¹ denotes hydrogen, methyl or ethyl, and

R² denotes alkyl with 1 to 23 carbon atoms, preferably alkyl with 12 to 23 carbon atoms, particularly preferably alkyl with 17 carbon atoms,

and in which

R³ and R⁴ (end groups) denote, independently of one another, hydrogen and alkyl with 1 to 24 carbon atoms, preferably alkyl with 13 to 24 carbon atoms, particularly preferably alkyl with 18 carbon atoms, or have a structure dependent on the initiator,

where

R⁵ (end group) is a substituent dependent on the termination reaction, for example hydroxyl, NH₂, NHR or NR₂, where the R radicals may correspond to the end groups R³ and R⁴,

and where the average degree of polymerization P=(m+n) is in the range from 45 to 5250, preferably in the range from 250 to 2250, particularly preferably in the range from 500 to 2050, and n=a×P with 0.001<a<0.1, preferably 0.01<a<0.05 and particularly preferably a=0.03.

In this case the units m and n are not blocked structures but are randomly distributed in the polymer.

Another polymer which can preferably be used for the complex formation has the following general formula:

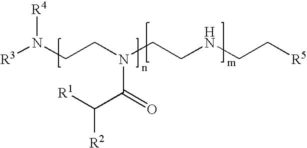

in which in each individual [CH₂—CH₂—N] unit

R¹ denotes hydrogen, methyl or ethyl, and

R² denotes alkyl with 1 to 22 carbon atoms, preferably alkyl with 11 to 22 carbon atoms, particularly preferably alkyl with 16 carbon atoms,

and in which

R³ and R⁴ (end groups) denote, independently of one another, hydrogen or acyl with 1 to 24 carbon atoms, preferably acyl with 13 to 24 carbon atoms, particularly preferably acyl with 18 carbon atoms, or have a structure dependent on the initiator,

where

R⁵ (end group) is a substituent dependent on the termination reaction, for example hydroxyl, NH₂, NHR or NR₂, where the R radicals may correspond to the end groups R³ and R⁴,

and where the average degree of polymerization P=(m+n) is in the range from 45 to 5250, preferably in the range from 250 to 2250, particularly preferably in the range from 500 to 2050, and n=a×P with 0.001<a<0.1, preferably 0.01<a<0.05 and particularly preferably a=0.03.

In this case the units m and n are not block structures but are randomly distributed in the polymer.

The polymer is novel and, as such, the present invention relates thereto.

Another polymer which can preferably be used for the complex formation has the general formula:

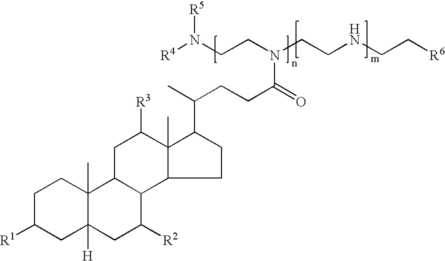

in which in each individual [CH₂—CH₂—N] unit

R¹, R² and R³ denote hydrogen or hydroxyl,

and in which

R⁴ and R⁵ (end groups) denote, independently of one another, hydrogen or bile acids, or have a structure dependent on the initiator,

where

R⁶ (end group) is a substituent dependent on the termination reaction, for example hydroxyl, NH₂, NHR or NR₂, where the R radicals may correspond to the end groups R⁴ and R⁵,

and where the average degree of polymerization P=(m+n) is in the range from 45 to 5250, preferably in the range from 250 to 2250, particularly preferably in the range from 500 to 2050, and n=a×P with 0.001<a<0.1, preferably 0.01<a<0.05 and particularly preferably a=0.03.

In this case the units m and n are not block structures but are randomly distributed in the polymer.

The polymer is novel and, as such, the present invention relates thereto. Moreover, also included are all stereoisomers in relation to the basic steroid framework. In particular, the substituents R¹, R² and R³ can be disposed both in the α and in the β configuration. The substituent in the 5 position may likewise be present in the α and in the β configuration (nomenclature according to Römpp-Chemie-Lexikon, 9^{th} edition, Georg Thieme Verlag, 1992).

Another polymer which can preferably be used for the complex formation has the following general formula:

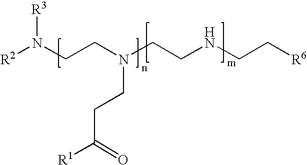

in which in each individual [CH₂—CH₂—N] unit

R¹ denotes OR⁴ or NR⁴R⁵,

where

R⁴ and R⁵ denote, independently of one another, hydrogen or alkyl with 1 to 24 carbon atoms, preferably alkyl with 13 to 24 carbon atoms, particularly preferably alkyl with 18 carbon atoms,

and in which

R² and R³ (end groups) independently of one another correspond to the substituents on the nitrogen atoms in the main polymer chain, or have a structure dependent on the initiator,

where

R⁶ (end group) is a substituent dependent on the termination reaction, for example hydroxyl, NH₂, NHR or NR₂, where the R radicals may correspond to the end groups R² and R³,

and where the average degree of polymerization P=(m+n) is in the range from 45 to 5250, preferably in the range from 250 to 2250, particularly preferably in the range from 500 to 2050, and n=a×P with 0.001<a<0.1, preferably 0.01<a<0.05 and particularly preferably a=0.03.

In this case the units m and n are not block structures but are randomly distributed in the polymer.

The polymer is novel and, as such, the present invention relates thereto.

Another polymer which can preferably be used for the complex formation has the following general formula:

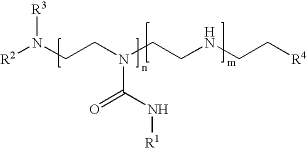

in which in each individual [CH₂—CH₂—N] unit

R¹ denotes alkyl with 1 to 24 carbon atoms, preferably alkyl with 13 to 24 carbon atoms, particularly preferably alkyl with 18 carbon atoms,

and in which

R² and R³ (end groups) independently of one another correspond to the substituents on the nitrogen atom in the main polymer chain, or have a structure dependent on the initiator,

where

R⁴ (end group) is a substituent dependent on the termination reaction, for example hydroxyl, NH₂, NHR or NR₂, where the R radicals may correspond to the end groups R² and R³,

and where the average degree of polymerization P=(m+n) is in the range from 45 to 5250, preferably in the range from 250 to 2250, particularly preferably in the range from 500 to 2050, and n=a×P with 0.001<a<0.1, preferably 0.01<a<0.05 and particularly preferably a=0.03.

In this case the units m and n are not block structures but are randomly distributed in the polymer.

The polymer is novel and, as such, the present invention relates thereto.

The polymer preferably has an average molecular weight below 220 000 g/mol, particularly preferably a molecular weight between 2000 and 100 000 g/mol, very particularly preferably a molecular weight between 20 000 and 100 000 g/mol.

The hydrophobic groups are inserted in polymer-analogous reactions, for example by alkylation with haloalkanes, acylation with carbonyl chlorides, acylation with reactive esters, Michael addition onto α,β-unsaturated carbonyl compounds (carboxylic acids, carboxamides, carboxylic esters) or by addition onto isocyanates. These are reaction types disclosed in the literature (J. March, Advanced Organic Chemistry, Wiley, New York, 4th edition, 1992).

The linear polyethyleneimines are prepared, for example, by cationic ring-opening polymerization of 2-ethyloxazoline with cationic initiators, preferably by a method of B. L. Rivas et al. (Polymer Bull. 1992, 28, 3-8). The poly(ethyloxazolines) obtained in this way are converted quantitatively into the linear polyethyleneimines, with elimination of propanoic acid, by treatment with a mixture of concentrated hydrochloric acid and water, preferably a 1:1 mixture of concentrated hydrochloric acid and water. The reaction temperature is preferably between 80 and 100° C., particularly preferably at 100° C. The reaction time is preferably between 12 and 30 hours, particularly preferably 24 hours. The product is purified preferably by recrystallization several times from ethanol.

It is possible with the described process to prepare the linear polyethyleneimines in the desired molecular weight range from 2000 to 220 000 g/mol.

The alkyl groups, such as, for example, C18 alkyl groups, are introduced for example by reacting a 5% strength solution of the appropriate linear polyethyleneimine in absolute ethanol at a reaction temperature of 40 to 75° C., preferably 60° C., with octadecyl chloride. The metered amount of alkyl chloride depends exactly on the desired degree of substitution (0.1 to 10%). The reaction time is preferably between 10 and 24 hours, particularly preferably 17 hours.

Acyl groups, such as, for example, C18 acyl groups, are introduced for example by reacting a 5% strength solution of the appropriate linear polyethyleneimine in absolute ethanol at a reaction temperature of 40 to 60° C., preferably 50° C., with octadecyl acid chloride. The metered amount of acid chloride depends exactly on the desired degree of substitution (0.01 to 10%). The reaction time is preferably between 10 and 24 hours, particularly preferably 20 hours.

Acyl groups can also be introduced by a reactive ester method with activation of a carboxylic acid derivative using N-hydroxysuccinimide. This process is preferably used in the case of functionalization of polyethyleneimine with bile acids. For this purpose, for example, the bile acid derivative chenodeoxycholic acid (3α,7α-dihydroxy-5β-cholanic acid), abbreviated hereinafter as substituent to CDC, is reacted with N-hydroxysuccinimide in dimethoxyethane as solvent in the presence of dicyclohexylcarbodiimide. The reaction takes place at room temperature, and the reaction time is 16 hours. The reactive ester prepared in this way is reacted with a 5% strength solution of the appropriate linear polyethyleneimine in absolute ethanol. The metered amount of the reactive ester depends exactly on the desired degree of substitution (0.01 to 10%). The reaction temperature is between 20 and 60° C., preferably at 50° C. The reaction time is preferably between 10 and 24 hours, particularly preferably 20 hours.

The introduction of, for example, chenodeoxycholic acid into oligoamines such as, for example, spermine or pentaethylenehexamine by the reactive ester method is described in the literature (S. Walker et al. Advanced Drug Delivery Reviews 1998, 30, 61-71.). The bile acid-substituted polymers according to the invention have hydrophobic substituents, it being possible to control the degree of hydrophobicity by the number of hydroxyl groups, in analogy to the “cationic facial amphiphiles” described by S. Walker et al.

Highly purified samples are preferably employed to prepare the complexes according to the invention. For this purpose, the hydrophobic linear polyethyleneimines are dissolved in a concentration of 0.1 to 1 mg/ml, preferably 0.5 mg/ml, in water at pH 7, and purified by a column chromatography on Sephadex and subsequent freeze drying. The polymers are then redissolved in water or, preferably, physiological saline with brief ultrasound treatment and adjusted to pH 7. The concentration of the polyethyleneimine solutions is preferably between 0.1 and 1 mg/ml, particularly preferably 0.5 mg/ml, for preparing the complexes.

It is possible to characterize the cationic polymers by using standard methods such as ¹H-NMR spectroscopy, FT-IR spectroscopy and zeta potential measurements.

The nucleic acid to be used for the complex formation can be, for example, a DNA or RNA. The nucleic acid can be an oligonucleotide or a nucleic acid construct. The nucleic acid preferably comprises one or more genes. The nucleic acid is particularly preferably a plasmid.

The nucleic acid may comprise a nucleotide sequence which codes for a pharmacological active substance or its precursor and/or which codes for an enzyme.

The nucleic acid may comprise a nucleotide sequence which codes for an antigen of a pathogen. Pathogens and relevant antigens belonging thereto are, for example: herpes simplex virus (HSV-1, HSV-2) and glycoprotein D; human immunodeficiency virus (HIV) and Gag, Nef, Pol; hepatitis C virus and NS3; anthrax and lethal factor, leishmania and ImSTI1 and TSA; tuberculosis bacteria and Mtb 8.4. It is possible in principle to employ any suitable nucleic acid which codes for an antigen against which there is an immune response. Diverse nucleic acids coding for antigens should be combined if necessary.

The nucleic acid may comprise a nucleotide sequence which codes for an allergen. Examples of allergens are f2 (house dust mite), Bet v1 (birch pollen), Ara h2 (peanut), Hev b5 (latex). It is possible in principle to employ any suitable nucleic acid which codes for an antigen which causes allergic reactions in humans or animals. Diverse nucleic acids coding for allergens should be combined if necessary.

The nucleic acid may comprise a nucleotide sequence which codes for an immunomodulatory protein. Examples of immunomodulatory proteins are cytokines (for example IL-4, IFNγ, IL-10, TNFα), chemokines (for example MCP-1, MIP1α, RANTES), costimulators (for example CD80, CD86, CD40, CD40L) or others (for example heat shock protein). CpG motifs in DNA sequences also display immunomodulatory properties.

The nucleic acid may, where appropriate, comprise a nucleotide sequence which codes for a fusion protein of antigen/allergen and immunomodulatory protein.

The nucleic acid preferably also comprises sequences which lead to a particular gene being expressed specifically, for example virus-specifically (that is to say, for example, only in virus-infected cells), (target) cell-specifically, metabolically specifically, cell cycle-specifically, development-specifically or else nonspecifically.

In the simplest case, the nucleic acid comprises a gene which encodes the desired protein, and specific promoter sequences and, where appropriate, other regulatory sequences. To enhance and/or prolong expression of the gene it is possible, for example, for viral promoter and/or enhancer sequences to be present. Such promoter and/or enhancer sequences are reviewed, for example, in Dion, TiBTech 1993, 11, 167. Examples thereof are the LTR sequences of Rous sarcoma viruses and of retroviruses, the promoter region and enhancer region of the CMV viruses, the ITR sequences and/or promoter sequences p5, p19 and p40 of AAV viruses, the ITR and/or promoter sequences of adenoviruses, the ITR and/or promoter sequences of vaccinia viruses, the ITR and/or promoter sequences of herpesviruses, the promoter sequences of parvoviruses and the promoter sequences (upstream regulator region) of papillomaviruses.

The complexes according to the invention may also comprise polymers to which cell-specific ligands are coupled. Such cell-specific ligands may be designed, for example, so that they bind to the outer membrane of a target cell, preferably an animal or human target cell. Ligand-containing complexes according to the invention can be used for target cell-specific transfer of a nucleic acid. The target cell can be, for example, an endothelial cell, a muscle cell, a macrophage, a lymphocyte, a glia cell, a blood-forming cell, a tumour cell, for example a leukemia cell, a virus-infected cell, a bronchial epithelial cell or a liver cell, for example a liver sinusoidal cell. A ligand which binds specifically to endothelial cells can be selected, for example, from the group consisting of monoclonal antibodies or fragments thereof which are specific for endothelial cells, mannose-terminated glycoproteins, glycolipids or polysaccharides, cytokines, growth factors, adhesion molecules or, in a particularly preferred embodiment, of glycoproteins from the envelope of viruses which have a tropism for endothelial cells. A ligand which binds specifically to smooth muscle cells can be selected, for example, from the group comprising monoclonal antibodies or fragments thereof which are specific for actin, cell membrane receptors and growth factors or, in a particularly preferred embodiment, of glycoproteins from the envelope of viruses which have a tropism for smooth muscle cells. A ligand which binds specifically to macrophages and/or lymphocytes can be selected, for example, from the group comprising monoclonal antibodies which are specific for membrane antigens on macrophages and/or lymphocytes, intact immunoglobulins or Fc fragments of polyclonal or monoclonal antibodies which are specific for membrane antigens on macrophages and/or lymphocytes, cytokines, growth factors, mannose-terminated peptides, proteins, lipids or polysaccharides or, in a particularly preferred embodiment, of glycoproteins from the envelope of viruses, in particular the HEF protein from Influenza C virus with mutation in nucleotide position 872 or HEF cleavage products of Influenza C virus containing the catalytic triads serine-71, histidine-368 or -369 and aspartic acid-261. A ligand which binds specifically to glia cells can be selected, for example, from the group comprising antibodies and antibody fragments which bind specifically to membrane structures of glia cells, adhesion molecules, mannose-terminated peptides, proteins, lipids or polysaccharides, growth factors or, in a particularly preferred embodiment, of glycoproteins from the envelope of viruses which have a tropism for glia cells. A ligand which binds specifically to blood-forming cells can be selected, for example, from the group comprising antibodies or antibody fragments which are specific for a receptor of the stem cell factor, IL-1 (in particular receptor type I or II), IL-3 (in particular receptor type α or β), IL-6 or GM-CSF, and intact immunoglobulins or Fc fragments which have this specificity, and growth factors such as SCF, IL-1, IL-3, IL-6 or GM-CSF and fragments thereof which bind to the relevant receptors. A ligand which binds specifically to leukemia cells can be selected, for example, from the group comprising antibodies, antibody fragments, immunoglobulins or Fc fragments which bind specifically to membrane structures on leukemia cells, such as CD13, CD14, CD15, CD33, CAMAL, sialosyl-Le, CD5, CD1e, CD23, M38, IL-2 receptors, T-cell receptors, CALLA or CD19, and growth factors or fragments derived therefrom or retinoids. A ligand which binds specifically to virus-infected cells can be selected, for example, from the group comprising antibodies, antibody fragments, intact immunoglobulins or Fc fragments which are specific for a viral antigen which is expressed on the cell membrane of the infected cell after infection by the virus. A ligand able to bind specifically to bronchial epithelial cells, liver sinusoidal cells or liver cells can be selected, for example, from the group comprising transferrin, asialoglycoproteins such as asialoorosomucoid, neoglycoproteins or galactose, insulin, mannose-terminated peptides, proteins, lipids or polysaccharides, intact immunoglobulins or Fc fragments which bind specifically to the target cells and, in a particularly preferred embodiment, of glycoproteins from the envelope of viruses which bind specifically to the target cells. Further detailed examples of ligands are disclosed, for example, in EP-A 0 790 312 and EP-A 0 846 772.

The invention further relates to the use of the complexes according to the invention. For example, the complexes can be used to introduce a nucleic acid into a cell or target cell (transfection), to produce a pharmaceutical and/or in gene therapy, and prophylactic and therapeutic vaccination and tolerance induction in the case of allergies. The invention preferably relates to the use of the complexes according to the invention for introducing nonviral or viral nucleic acid constructs into a cell and to the administration of this (transfected) cell to a patient for the purpose of prophylaxis or therapy of a disease, it being possible for the cell to be, for example, an endothelial cell, a lymphocyte, a macrophage, a liver cell, a fibroblast, a muscle cell or an epithelial cell, and it being possible for this cell to be applied locally onto the skin or injected subcutaneously, intramuscularly, into a wound, into a body cavity, into an organ or into a blood vessel. In another preferred embodiment, the invention relates to the use of the complexes according to the invention for the prophylaxis or therapy of a disease, it being possible to administer the complexes according to the invention in a conventional way, preferably orally, parenterally or topically. The complexes according to the invention can be given or injected for example perlingually, intranasally, dermally, subcutaneously, intravenously, intramuscularly, rectally, into a wound, into a body cavity, into a body orifice, into an organ or into a blood vessel.

It may be worthwhile where appropriate to combine the complexes according to the invention with further additions (adjuvants, anesthetic etc.).

One advantage of the complexation according to the invention of nucleic acids before introduction into the patient is based on the fact that the formation of anti-DNA antibodies is made difficult thereby. Naked DNA introduced into experimental animals by contrast led in lupus-prone mice to an increase in the formation of autoimmune antibodies and a tripling of the number of auto-antibody secreting B cells (Klinman et al., DNA vaccines: safety and efficacy issues, in Gene Vaccination: Theory and Practice, ed. E. Raz, Springer).

The present invention further relates to a process for producing a transfected cell or target cell, where the complexes according to the invention are incubated with this cell. The transfection is preferably carried out in vitro. The invention further relates to a transfected cell or target cell which contains the complexes according to the invention. The invention further relates to the use of the transfected cell, for example as pharmaceutical or for producing a pharmaceutical and/or for gene therapy.

The present invention further relates to a pharmaceutical which contains the complexes according to the invention and/or a cell transfected therewith.

The present invention also relates to a process for producing a pharmaceutical, where the complexes according to the invention are mixed with other additives.

The present invention also relates to the coupling of the polymers according to the invention to a cell-specific ligand and to the use of the coupling product in a complex with a viral or nonviral nucleic acid for introducing this nucleic acid into a cell or for administering the complex to a mammal for the prophylaxis or therapy of a disease. The possibilities for producing and coupling cell-specific ligands has already been described in detail in the patent applications EP-A 0 790 312 and DE-A 196 49 645. Express reference is made to these patent applications.

The complexes according to the invention of polymer, where appropriate coupled to a cell-specific ligand, and of a viral or nonviral nucleic acid construct represent a gene transfer material for gene therapy. In a preferred embodiment, these complexes are administered to patients externally or internally, locally, into a body cavity, into an organ, into the bloodstream, into the respiratory tract, into the gastrointestinal tract, into the urogenital tract or orally, intranasally, intramuscularly or subcutaneously.

The present invention also relates to cells, in particular from yeasts or mammals, into which a nucleic acid construct has been introduced with the aid of the complexes according to the invention. In a particularly preferred embodiment, the nucleic acid constructs are introduced with the aid of the complexes according to the invention into cell lines which can then be used after transfection for expression of the chosen gene. These cells can thus be used to provide a pharmaceutical for patients.

The invention further relates to the use of mammalian cells into which a nucleic acid has been introduced with the aid of the complexes according to the invention for producing a pharmaceutical for the treatment or prophylaxis of a disease. For example, endothelial cells can be obtained from the blood, be treated in vitro with the complexes according to the invention and be injected, for example intravenously, into the patient. A further possibility is, for example, for dendritic cells (antigen-presenting cells) to be obtained from blood, be treated in vitro with the complexes according to the invention and be injected into the patient to induce a prophylactic or therapeutic immune response. Such cells transfected in vitro can also be administered to patients in combination with the complexes according to the invention. This combination comprises cells and complexes being administered or injected in each case simultaneously or at different times, at the same or at different sites.

The polymers according to the invention are complexed with the nucleic acid by mixing the two starting substances. The mixing ratio is determined by the desired charge ratio between negatively charged nucleic acid and positively charged polymer. It has been possible to establish from zeta potential measurements that in the case of the linear polyethyleneimines with hydrophobic functionalities (H-LPEI) the degree of protonation at pH 7 is about 50%. The DNA/polymer charge ratio may vary between 1:0.1 and 1:10. The preferred charge ratio is between 1:2 and 1:10. With charge ratios of 1:5 to 1:10 turbidity or precipitation may occur at a DNA concentration of 100 μg/ml. If precipitates are produced they can be resuspended or redispersed before administration.

The complexes according to the invention are preferably produced by adding the H-LPEI solution to the appropriate nucleic acid solution. The concentrations are particularly preferably adjusted so that a 1:1 mixture by volume is produced.

The complexes can be examined by agarose gel electrophoresis in order to characterize the charge ratios. Selected complexes can be examined by scanning force microscopy in order to obtain information about the DNA condensation and the size of the complexes.

It is surprising that, in particular, hydrophobic groups bound to the polymer chain show, despite reduced solubility in water, particularly good results and form defined condensed complexes. It was necessarily expected that polymers with hydrophobic modifications act like surfactants or emulsifiers and therefore are unable to form particulate complexes with nucleic acids. It was further to be expected that the hydrophobic substituents determine the surface characteristics of the nucleic acid/polymer complexes, which consequently leads to an increased interaction with cell membranes and thus to an increased transfection efficiency.

### EXAMPLES

General

It has surprisingly emerged that the hydrophobic linear polyethyleneimines, abbreviated to H-LPEI hereinafter, are distinctly superior in respect of efficacy as vector for introducing nucleic acids into cells and in its biocompatibility to linear unsubstituted polyethyleneimines (LPEI). In experiments on mice, nucleic acid complexes containing H-LPEI and DNA plasmid which encodes the human factor VIII (FVIII) protein were tested in comparison with linear unsubstituted polyethyleneimines of the same molecular weight in each case. Protein expression was detectable only in the case of the H-LPEI complexes. Likewise, transfection experiments with naked DNA were always negative.

In the investigations on FVIII gene therapy, acylated polyethyleneimines in particular proved to be effective, preferably with a C18 side chain. The degree of acylation is between 0.1 and 10%, preferably between 1 and 5%, and particularly preferably 3%. The average molecular weight is preferably in the range from 20 000 to 100 000 g/mol.

In addition, in particular linear polyethyleneimines with bile acid substituents were identified as effective, preferably with CDC substituents. The degree of acylation is between 0.1 and 10%, preferably between 1 and 5%, and particularly preferably 3%. The molecular weight is preferably in the range from 20 000 to 100 000 g/mol.

At the same time, no toxic reactions were observed during the in vivo tests.

The analysis and the determination of FVIII protein expression in the in vivo experiments, and the corresponding protocols, are described in detail in the following examples.

### Example 1

Synthesis of the Linear Polyethyleneimines (LPEI):

Linear polyethylenes were synthesized by cationic ring-opening polymerization of 2-ethyloxazoline to poly(ethyloxazoline) (in analogy to B. L. Rivas, S. I. Ananias, Polymer Bull. 1992, 28, 3-8) and subsequent acidic hydrolysis through elimination of propanoic acid. Certain precursor polymers (poly(ethyloxazolines)) are also commercially available (Sigma-Aldrich Chemie GmbH, Germany). The precursor polymers were characterized by gel permeation chromatography, ¹H-NMR and FT-IR.

Quantitative hydrolysis was possible by reacting, for example, 24.7 g of poly(ethyloxazoline) (MW 200 000 g/mol) in a mixture of 40 ml of water and 40 ml of concentrated hydrochloric acid at 100° C. The voluminous precipitate which had formed after 24 hours was dissolved by adding 250 ml of water. After cooling to 20° C., the product was adjusted to pH 11 by adding 20% strength NaOH and was precipitated. The precipitate was filtered off with suction and washed (wash water pH 7) and then dried under high vacuum over phosphorus pentoxide. The crude product was then recrystallized from ethanol (yield 9.5 g/88%). High-purity batches (milligramme quantities) were obtained by column chromatography on Sephadex G25 (Pharmacia disposable PD-10 desalting column) from saturated aqueous solutions (pH 7) of the polyethyleneimine with Millipore water as eluent and subsequent freeze drying.

The linear polyethyleneimines were characterized by ¹H-NMR and Fr-IR, by which means it was possible to confirm the quantitative hydrolysis.

### Example 2

Synthesis of the Linear Polyethyleneimines with Hydrophobic Functionalities (H-LPEI) Taking the Example of the Introduction of 3 mol % C18 Alkyl Groups into LPEI with an MW of 87 000 g/mol:

For this purpose, 0.5 g of LPEI was dissolved in 10 ml of ethanol at 60° C. under argon and, after slow addition of 0.11 g (0.13 ml) of octadecyl chloride, stirred for 17 hours. The reaction product was precipitated by adding 20 ml of water at 20° C. and was filtered off, washed with water (wash water pH 7) and dried under high vacuum over phosphorus pentoxide (yield 0.48 g/96%). High-purity batches (milligramme quantities) were obtained by column chromatography on Sephadex G25 (Pharmacia disposable PD-10 desalting column) from saturated aqueous solutions (pH 7) of the polyethyleneimine with Millipore water as eluent and subsequent freeze drying.

The alkylated linear polyethyleneimines were characterized by ¹H-NMR and FT-IR, by which means it was possible to confirm the desired degree of alkylation.

### Example 3

Synthesis of the Linear Polyethyleneimines with Hydrophobic Functionalities (H-LPEI) Taking the Example of the Introduction of 3 mol % C18 Acyl Groups into LPEI with an MW of 87 000 g/mol:

For this purpose, 0.5 g of LPEI was dissolved in 10 ml of ethanol at 50° C. under argon and, after slow addition of 0.11 g (0.12 ml) of octadecanoyl chloride, stirred for 20 hours. The reaction mixture was filtered and then quantitatively concentrated in vacuo. The residue was dissolved in 4 ml of hot ethanol and the product was precipitated by adding 8 ml of water at 20° C. Filtration and washing with water (wash water pH 7) were followed by drying under high vacuum over phosphorus pentoxide (yield 0.38 g/76%). High-purity batches (milligramme quantities) were obtained by column chromatography on Sephadex G25 (Pharmacia disposable PD-10 desalting column) from saturated aqueous solutions (pH 7) of the polyethyleneimine with Millipore water as eluent and subsequent freeze drying.

The acylated linear polyethyleneimines were characterized by ¹H-NMR and FT-IR, by which means it was possible to confirm the desired degree of acylation.

### Example 4

Synthesis of the Linear Polyethyleneimines with Hydrophobic Functionalities (H-LPEI) Taking the Example of the Introduction of 3 mol % Chenodeoxycholic Acid Groups (3α,7α-dihydroxy-5β-cholanic acid) into LPEI with an MW of 87 000 g/mol:

Chenodeoxycholic acid (Sigma-Aldrich Chemie GmbH) was for this purpose converted into a reactive ester compound with N-hydroxysuccinimide. 1 g of chenodeoxycholic acid and 0.32 g of N-hydroxysuccinimide were dissolved in 5 ml of dimethoxyethane and, at 0-5° C., reacted with 0.63 g of dicyclohexylcarbodiimide. The reaction mixture was stirred for 16 hours, the precipitate was filtered off, and the filtrate was concentrated in vacuo. The reactive ester was dried under high vacuum (stable foam) and characterized by ¹H-NMR. Without further purification, 179 mg of the chenodeoxycholic acid reactive ester were added to a solution of 0.5 g of LPEI in 10 ml of ethanol at room temperature under argon. The reaction mixture was then stirred at 50° C. for 20 hours. After cooling to room temperature, the product was precipitated by adding 25 ml of water. The residue was filtered off, washed with water (wash water pH 7) and dried under high vacuum over phosphorus pentoxide (yield 0.41 g/82%). High-purity batches (milligramme quantities) were obtained by column chromatography on Sephadex G25 (Pharmacia disposable PD-10 desalting column) from saturated aqueous solutions (pH 7) of the polyethyleneimine with Millipore water as eluent and subsequent freeze drying.

The linear polyethyleneimines which have been acyl-functionalized by the reactive ester method were characterized by ¹H-NMR and FT-IR, by which means it was possible to confirm the desired degree of acylation.

### Example 5

Zeta Potential Measurements:

Zeta potential measurements were carried out to establish the charge and the degree of protonation of the linear polyethyleneimines and of the polyethyleneimines with hydrophobic functionalities in aqueous solution at a physiological pH. Irrespective of the average molecular weight and irrespective of the polymer type, the average degree of protonation at pH 7 was found to be 50%, that is to say about 50% of the nitrogen atoms are in protonated form in aqueous solution at pH 7.

### Example 6

Preparation of the Polynucleotide/Polymer Complexes:

The aim was to produce polynucleotide/polymer complexes taking the example of the complexation of the FVIII plasmid pCY2 with various polynucleotide/polymer charge ratios (1:0.1 to 1:10) and a constant polynucleotide concentration of 250 μg/ml. The charge ratios and the corresponding concentrations can be calculated on the basis of the zeta potential measurements presented in Example 5.

The plasmid pCY2 is described in the literature (C. R. Ill, C. Q. Yang, S. M. Budlingmaier, J. N. Gonzales, D. S. Burns, R. M. Bartholomew and P. Scuderi, Blood Coagulation and Fibrinolysis 1997, 8(2), 23-30). PCY2 is 9164 bp long and contains the thyroid hormone binding globulin promoter, two copies of the alpha-i micro-globulin/bikunin enhancer and the 5′ region of a rabbit beta-globulin gene intron which controls expression of a human B region-deleted FVIII gene. The plasmid also contains an ampicillin antibiotic resistance gene, the ColEl origin of replication and a polyA site.

Stock solutions were produced of all the polyethyleneimines (LPEI, H-LPEI) both in water and in physiological saline at pH 7 with a concentration of 0.5 mg/ml. This was done by dissolving 25 mg of the LPEI or of the H-LPEI in 30 ml of water or physiological saline with heating and brief ultrasound treatment, adjusting to pH 7 with 0.1 N HCl and making up to a final volume of 50 ml. The stock solutions were sterilized by filtration (0.2 μm) and can be stored for a long time at 20° C. Serial dilutions were prepared (1 ml each, Table 1) from the stock solutions and were reacted with polynucleotide solutions of a concentration of 500 μg/ml in the ratio 1:1 by volume to result in a polynucleotide complex with a defined charge and polynucleotide concentration of 250 μg/ml (Table 2). In standard experiments, a volume of 300 μl of the polynucleotide/LPEI or polynucleotide/H-LPEI solution was frequently chosen. Precipitates may occur with complexes having a high polyethyleneimine content and can be resuspended or redispersed before the particular application.

The polymer solutions were pipetted into the polynucleotide solutions at room temperature under sterile conditions and then mixed in a Vortex. After an incubation time of 4 hours at room temperature, the polynucleotide/polymer complexes were stored at 4° C., the complexes being stable on storage for several weeks. The complex solutions can be diluted as required for the animal experiments.

TABLE 1
Preparation of serial dilutions from LPEI and H-LPEI stock solutions

| | LPEI, H-LPEI | | |
|---|---|---|---|
| | stock solution | Water or phys. | |
| LPEI, H-LPEI | c = 500 μg/ml | saline | Total volume |
| c/μg/ml | V/μl | V/μl | V/μl |
| 19 | 38 | 962 | 1000 |
| 47 | 95 | 905 | 1000 |
| 95 | 189 | 811 | 1000 |
| 142 | 284 | 716 | 1000 |
| 189 | 378 | 622 | 1000 |
| 378 | 756 | 244 | 1000 |

TABLE 2
Summary of the preparation of
polynucleotide/LPEI and H-LPEI
complexes (aqueous solutions) with various charge
ratios for in vivo experiments and for the
investigations by gel electrophoresis

| Poly- | | | | | | |
|---|---|---|---|---|---|---|
| nucleo- | | | Water | Poly- | Poly- | |
| tide/- | | | or | nucleotide | nucleotide | |
| polymer | | | phys. | c = | c = | |
| charge | LPEI/H-LPEI | | saline | 500 μg/ml | 1000 μg/ml | Complex |
| ratio | c/μg/ml | V/μl | V/μl | V/μl | V/μl | Vₜₒₜₐₗ/μl |
| 1:01 | 19 | 150 | 0 | 150 | 0 | 300 |
| 1:0.25 | 47 | 150 | 0 | 150 | 0 | 300 |
| 1:0.5 | 95 | 150 | 0 | 150 | 0 | 300 |
| 1:0.75 | 142 | 150 | 0 | 150 | 0 | 300 |
| 1:1 | 189 | 150 | 0 | 150 | 0 | 300 |
| 1:2 | 378 | 150 | 0 | 150 | 0 | 300 |
| 1:3 | 500 | 170 | 55 | 0 | 75 | 300 |

### Example 7

Characterization of the Polynucleotide/Polymer Complexes by Gel Electrophoresis:

The complexation behaviour of the polymers and the charge situation of the polynucleotide/polymer complexes was investigated by agarose gel electrophoresis. The gels were each prepared from 0.4 g of agarose and 40 ml of tris acetate buffer (0.04 M, pH 8.3 with 0.01 M EDTA) (thickness about 0.6 cm). Samples consisting of 4 μl of polynucleotide/polymer complex (c=250 μg/ml), 9.5 μl of water (Millipore) and 1.5 μl of stop mix were mixed in a Vortex and transferred quantitatively into the gel pockets. The gel electrophoresis usually took place with a current of 100 to 150 mA (110 V). For comparison, a DNA marker (PeqLab, 1 kb Ladder) and naked (uncomplexed) polynucleotide were also analysed in each gel electrophoresis run.

After development of the gel in an aqueous solution of ethidium bromide and irradiation at 254 nm, the location of the DNA bands was visualized. In the case of the FVIII plasmid, 2 bands are visible, corresponding to the supercoiled and the circular form of the plasmid, and migrating in the direction of the anode. LPEI and H-LPEI were undetectable with ethidium bromide. An increase in polymer content in the complexes lead to a partial but still incomplete retardation of the plasmid at the loading point. Complexes with a polynucleotide/polymer charge ratio above 1:1 were no longer detectable, that is to say intercalation of ethidium bromide into the DNA was no longer possible. It is to be assumed that the compacted DNA is in the form of polymer-encapsulated particles above a charge ratio of 1:1. The results of the gel electrophoresis do not depend on the type (molecular weight, substitution) of the linear polyethyleneimines investigated. The calculated charge ratios (see Example 5) can be confirmed by gel electrophoresis.

### Example 8

Characterization of the Polynucleotide/Polymer Complexes by Scanning Force Microscopy (AFM):

Selected polynucleotide/polymer complexes prepared in aqueous solution were characterized by AFM (Digital Instruments). For this purpose, the solutions of the complexes were diluted to a concentration of 0.5 to 1 μg/ml with water, and between 1 and 5 μl of the diluted solutions were pipetted onto a silicon substrate. After evaporation of the water (about 5 min), the sample is analysed in the AFM. It was possible to show that above a polynucleotide/polymer ratio of 1:0.15 there is DNA condensation and particle formation, the size of the particles being in the range from 100 to 200 nm.

### Example 9

In Vivo Transfection Experiments with Polyethyleneimines with Hydrophobic Functionalities (H-LPEI):

The polynucleotide/polymer complexes were produced using the plasmid pCY2 coding for FVIII.

The mice used were C57B1/6 female mice, 5-6 weeks old and approximately 20 g each. The mice were purchased from Simonsen Labs Inc, USA.

In the experiments, 5 mice/group were used and were injected 200 μl/animal via the tail vein with either 50 μg of plasmid DNA alone or 50 μg plasmid DNA+polymer. The DNA/polymer charge ratio was 1:0.5. Subsequent experiments used 10 mice/group and different charge ratios of DNA: polymer/LPEI and polymer/H-LPEI, respectively. The animals were retro-orbitally bled 24 hrs post-injection.

Plasma samples from these animals were assayed using a modified FVIII activity assay. The plasma was first diluted 1:4 in phosphate buffered saline prior to addition to a 96-well assay plate coated with murine monoclonal antibody C7F7. The C7F7 antibody is specific for the light chain of human FVIII and does not react with murine FVIII. After a 2-hr incubation at 37° C., the plate was washed twice with PBS containing 0.05% Tween 20. Subsequently reagents and assay conditions specified by the manufacturer of the Coatest kit (Diapharma Inc., Sweden) were used. The final step in the assay was an optical density reading taken at 405/450 nm. All FVIII levels were extrapolated from a standard curve made by adding recombinant human FVIII to diluted mouse plasma (calibration shown in Table 3).

The results are shown in Tables 4 and 5.

FVIII activity assay (C7F7 modified Coatest):

Reagents and Buffers:

Coating Buffer: either Sigma P-3813, pH 7.4 or 0.1 M bicarbonate buffer pH 9.2;

Blocking Buffer: 1× Coatest buffer solution+0.8% BSA+0.05% Tween 20;

Wash Buffer: 20 mM tris-HCl, 0.1 M NaCl, 0.05% Tween 20 pH 7.2 filter before use;

Incubation Buffer: blocking buffer without Tween 20;

Coatest VIII: C/4 assay kit: Chromogenix AB, #82-19-18-63/2

Procedure:

1. Coat a 96-well Immulon plate with 5 μg/ml C7F7 in coating buffer (100 μl/well) overnight at 4° C.;

2. Wash×3; add blocking buffer (100 μl/well); incubate at least 1 hr at 37° C.;

3. Wash×3; add samples diluted in blocking buffer (100 μl/well); incubate 1-2 hours at 37° C.;

4. Wash×3; add incubation buffer (25 μl/well); followed by Coatest reagents (kit: 50 μl/well of mixed FIXa, FX+phospholipid); follow the kit's mixing instructions; incubate 5 minutes at 37° C.; then add 50 μl of substrate S-222 to each well and incubate 5 minutes at 37° C., or 10 minutes for lower range values (Step 4 may be done in a heated block with shaker);

5. Stop reaction with 2% citric acid (50 μl/well);

6. Measure O.D. at 405-450 nm.

### Example 10

Comparative Experiments (Table 5a,b):

In vivo comparative experiments with the naked FVIII plasmid pCY2 were always negative, that is to say no protein expression was detectable. In comparative experiments with plasmid/polymer complexes based on unsubstituted linear polyethyleneimines (LPEI) with three different molecular weight distributions (MW 22 000, 87 000, 217 000 g/mol) and a plasmid/LPEI charge ratio of, for example, 1:0.5 (IV injection of 200 μl, c=250 μg/ml based on DNA) it was likewise impossible to detect any protein expression.

TABLE 3
UV/vis spectroscopic calibration of the FVIII protein standards
(duplicate determination)

| | FVIII conc./ | Position | Optical density | |
|---|---|---|---|---|
| Standard | ng/ml | (MTP format) | (O.D.) | Mean O.D. |
| STD01 | 23.00 | A1 | 1.289 | 1.289 |
| | | A2 | 1.149 | |
| STD02 | 11.50 | B1 | 1.037 | 0.993 |
| | | B2 | 0.949 | |
| STD03 | 5.750 | C1 | 0.687 | 0.652 |
| | | C2 | 0.617 | |
| STD04 | 2.875 | D1 | 0.456 | 0.43 |
| | | D2 | 0.404 | |
| STD05 | 1.438 | E1 | 0.293 | 0.277 |
| | | E2 | 0.261 | |
| STD06 | 0.719 | F1 | 0.182 | 0.171 |
| | | F2 | 0.160 | |
| STD07 | 0.359 | G1 | 0.121 | 0.117 |
| | | G2 | 0.114 | |
| STD08 | 0.179 | H11 | 0.104 | 0.110 |
| | | H12 | 0.115 | |
| STD09 | 0.000 | H1 | 0.058 | 0.059 |
| | | H2 | 0.060 | |

TABLE 4a
FVIII gene expression after injection of DNA/polymer complexes:
Group 1, 5 mice (1a-1e), polymer: H-LPEI, MW 86 980, C18, acyl,
3 mol % (*dilution factor 4)

| | Optical density | | FVIII conc./ | FYIII conc./ng/ml |
|---|---|---|---|---|
| Group 1 | (O.D.)* | Mean O.D. | ng/ml | (Mean) |
| 1a | 0.219 | 0.198 | 3.435 | 2.950 |
| | 0.177 | | 2.464 | |
| 1b | 0.075 | 0.079 | 0.221 | 0.298 |
| | 0.082 | | 0.376 | |
| 1c | 0.075 | 0.075 | 0.221 | 0.210 |
| | 0.074 | | 0.198 | |
| 1d | 0.090 | 0.085 | 0.551 | 0.430 |
| | 0.079 | | 0.310 | |
| 1e | 0.070 | 0.071 | 0.107 | 0.119 |
| | 0.071 | | 0.130 | |

TABLE 4b
FVIII gene expression after injection of DNA/polymer complexes:
Group 2, 5 mice (2a-2e), polymer: H-LPEI, MW 86 980, CDC,
3 mol % (*dilution factor 4)

| | Optical density | | FVIII conc./ | FVIII conc./ng/ml |
|---|---|---|---|---|
| Group 2 | (O.D.)* | Mean O.D. | ng/ml | (Mean) |
| 2a | 0.066 | 0.066 | <<<<< | 0 |
| | 0.065 | | <<<<< | |
| 2b | 0.076 | 0.077 | 0.243 | 0.265 |
| | 0.078 | | 0.288 | |
| 2c | 0.067 | 0.064 | <<<<< | 0 |
| | 0.061 | | <<<<< | |
| 2d | 0.076 | 0.073 | 0.243 | 0.175 |
| | 0.070 | | 0.107 | |
| 2e | 0.087 | 0.082 | 0.485 | 0.364 |
| | 0.076 | | 0.242 | |

TABLE 5a
FVIII gene expression after injection of naked DNA: Group 3, 5 mice
(DNA1-DNA5), (*dilution factor 4)

| | | | | FVIII conc./ |
|---|---|---|---|---|
| | Optical density | | FVIII conc./ | ng/ml |
| Group 3 | (O.D.)* | Mean O.D. | ng/ml | (Mean) |
| DNA1 | 0.065 | 0.063 | <<<<< | 0 |
| | 0.062 | | <<<<< | |
| DNA2 | 0.063 | 0.061 | <<<<< | 0 |
| | 0.059 | | <<<<< | |
| DNA3 | 0.056 | 0.057 | <<<<< | 0 |
| | 0.058 | | <<<<< | |
| DNA4 | 0.062 | 0.062 | <<<<< | 0 |
| | 0.062 | | <<<<< | |
| DNA5 | 0.065 | 0.065 | <<<<< | 0 |
| | 0.065 | | <<<<< | |

TABLE 5b
FVIII gene expression after injection of DNA/polymer complexes:
Group 4, 5 mice (4a-4e), polymer: LPEI, MW 86 980 g/mol,
unsubstituted (*dilution factor 4)

| | | | | FVIII conc./ |
|---|---|---|---|---|
| | Optical density | | FVIII conc./ | ng/ml |
| Group 4 | (O.D.)* | Mean O.D. | ng/ml | (Mean) |
| 4a | 0.063 | 0.061 | <<<<< | 0 |
| | 0.059 | | <<<<< | |
| 4b | 0.059 | 0.059 | <<<<< | 0 |
| | 0.060 | | <<<<< | |
| 4c | 0.066 | 0.065 | <<<<< | 0 |
| | 0.064 | | <<<<< | |
| 4d | 0.069 | 0.068 | <<<<< | 0 |
| | 0.067 | | <<<<< | |
| 4e | 0.089 | 0.086 | <<<<< | 0.412 |
| | 0.082 | | <<<<< | |

### Example 11

In order to test the behaviour of the polynucleotide/polymer complexes when the pH changes and thus to simulate the effect of the endosomal-lysosomal compartment of the cell, agarose gel electrophoresis studies were carried out in various buffer systems and thus under variable pH conditions. It was possible to show that the degree of complexation decreases on changing from pH 8.3 (TAE buffer) to pH 5.9 (MES buffer), which is equivalent to partial release.

1. Complex comprising a linear cationic polymer which is soluble or dispersible in water and has hydrophobic substituents, and at least one nucleic acid.

2. Complex according to claim 1, characterized in that the polymer is a polyamine.

3. Complex according to claim 2, characterized in that the polyamine is a polyethyleneimine.

4. Complex according to any of claims 1 to 3, characterized in that the substituents are disposed as side chains or terminally on the polymer.

5. Complex according to any of claims 1 to 4, characterized in that the substituents are alkyl chains, acyl chains or steroid-like substituents, and hydrophobic substituents which can be introduced by addition of the nitrogen functions of the main polymer chain onto isocyanates or onto α,β-unsaturated carbonyl compounds.

6. Complex according to any of claims 1 to 5, characterized in that the polymer has the following general formula: 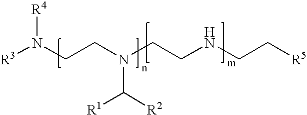
in which in each individual [CH₂—CH₂—N] unit
R¹ denotes hydrogen, methyl or ethyl, and
R² denotes alkyl with 1 to 23 carbon atoms,
and in which
R³ and R⁴ (end groups) denote, independently of one another, hydrogen and alkyl with 1 to 24 carbon atoms, or have a structure dependent on the initiator,
where
R⁵ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

7. Complex according to any of claims 1 to 5, characterized in that the polymer has the following general formula: 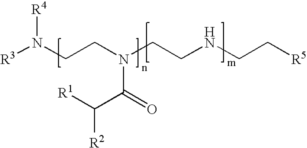
in which in each individual [CH₂—CH₂—N] unit
R¹ denotes hydrogen, methyl or ethyl, and
R² denotes alkyl with 1 to 22 carbon atoms,
and in which
R³ and R⁴ (end groups) denote, independently of one another, hydrogen or acyl with 1 to 24 carbon atoms, or have a structure dependent on the initiator,
where
R⁵ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

8. Complex according to any of claims 1 to 5, characterized in that the polymer has the following general formula: 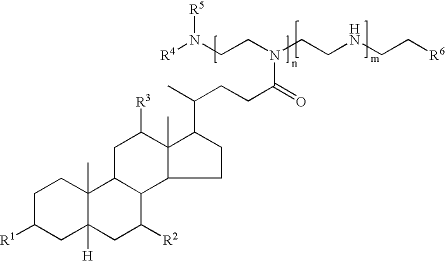
in which in each individual [CH₂—CH₂—N] unit
R¹, R² and R³ denote hydrogen or hydroxyl,
and in which
R⁴ and R⁵ (end groups) denote, independently of one another, hydrogen or bile acids, or have a structure dependent on the initiator,
where
R⁶ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

9. Complex according to any of claims 1 to 5, characterized in that the polymer has the following general formula: 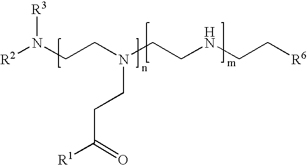
in which in each individual [CH₂—CH₂—N] unit
R¹ denotes OR⁴ or NR⁴R⁵,
where
R⁴ and R⁵ denote, independently of one another, hydrogen or alkyl with 1 to 24 carbon atoms,
and in which
R² and R³ (end groups) independently of one another correspond to the substituents on the nitrogen atoms in the main polymer chain, or have a structure dependent on the initiator,
where
R⁶ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

10. Complex according to any of claims 1 to 5, characterized in that the polymer has the following general formula: 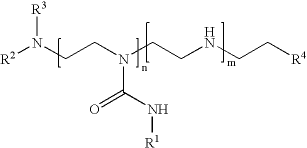
in which in each individual [CH₂—CH₂—N] unit
R¹ denotes alkyl with 1 to 24 carbon atoms,
and in which
R² and R³ (end groups) independently of one another correspond to the substituents on the nitrogen atom in the main polymer chain, or have a structure dependent on the initiator,
where
R⁴ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

11. Complex according to any of claims 1 to 10, characterized in that the polymer has an average molecular weight below 220 000 g/mol.

12. Complex according to any of claims 1 to 11, characterized in that the polymer has a molecular weight from 2000 to 100 000 g/mol.

13. Complex according to any of claims 1 to 12, characterized in that the polymer is coupled to a cell-specific ligand.

14. Complex according to any of claims 1 to 13, characterized in that the nucleic acid is a plasmid.

15. Complex according to any of claims 1 to 14, characterized in that the nucleic acid comprises a nucleotide sequence which codes for a pharmacological active substance.

16. Complex according to any of claims 1 to 14, characterized in that the nucleic acid comprises a nucleotide sequence which codes for an antigen, allergen or immunomodulatory protein.

17. Complex according to any of claims 1 to 16, characterized in that the nucleic acid/polymer charge ratio is between 1:0.1 and 1:10, in particular between 1:2 and 1:10.

18. Process for the production of a complex according to any of claims 1 to 17, characterized in that an appropriate amount of the polymer present in aqueous solution is mixed with an appropriate amount of a nucleic acid solution.

19. Process according to claim 18, characterized in that the mixture is then dried.

20. Complex according to any of claims 1 to 16 for use as pharmaceutical.

21. Composition containing a complex according to any of claims 1 to 16 and further additives.

22. Use of a complex according to any of claims 1 to 16 for introducing a nucleic acid into a cell.

23. Cell containing a complex according to any of claims 1 to 16.

24. Composition containing a cell according to claim 23 and further additives.

25. Use of a complex according to any of claims 1 to 16 for producing a pharmaceutical for gene therapy.

26. Use of a complex according to any of claims 1 to 16 for producing a pharmaceutical for vaccination.

27. Use of a complex according to any of claims 1 to 16 for producing a pharmaceutical for tolerance induction in the case of allergies.

28. Polymer of the general formula 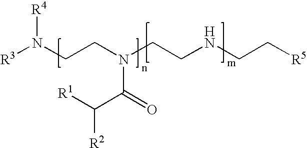
in which in each individual [CH₂—CH₂—N] unit
R¹ denotes hydrogen, methyl or ethyl, and
R² denotes alkyl with 1 to 22 carbon atoms,
and in which
R³ and R⁴ (end groups) denote, independently of one another, hydrogen or acyl with 1 to 24 carbon atoms, or have a structure dependent on the initiator,
where
R⁵ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

29. Polymer of the general formula 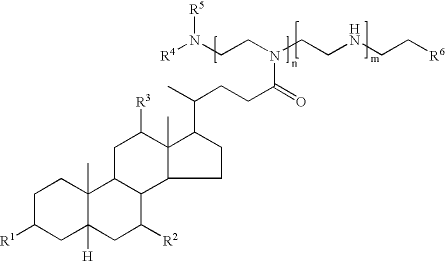
in which in each individual [CH₂—CH₂—N] unit
R¹, R² and R³ denote hydrogen or hydroxyl,
and in which
R⁴ and R⁵ (end groups) denote, independently of one another, hydrogen or bile acids, or have a structure dependent on the initiator,
where
R⁶ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

30. Polymer of the general formula 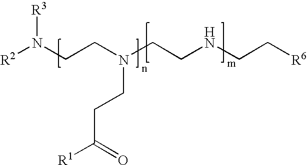
in which in each individual [CH₂—CH₂—N] unit
R¹ denotes OR⁴ or NR⁴R⁵,
where
R⁴ and R⁵ denote, independently of one another, hydrogen or alkyl with 1 to 24 carbon atoms,
and in which
R² and R³ (end groups) independently of one another correspond to the substituents on the nitrogen atoms in the main polymer chain, or have a structure dependent on the initiator,
where
R⁶ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

31. Polymer of the general formula 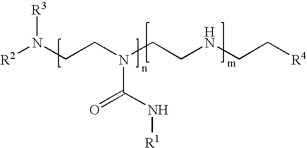
in which in each individual [CH₂—CH₂—N] unit
R¹ denotes alkyl with 1 to 24 carbon atoms,
and in which
R² and R³ (end groups) independently of one another correspond to the substituents on the nitrogen atom in the main polymer chain, or have a structure dependent on the initiator,
where
R⁴ (end group) is a substituent dependent on the termination reaction,
and where the average degree of polymerization P=(m+n) is in the range from 45 to 5250, and n=a×P with 0.001<a<0.1, where the units m and n are randomly distributed in the polymer.

32. Polymer according to any of claims 28 to 31, characterized in that it has a molecular weight below 220 000 g/mol.

33. Polymer according to claim 32, characterized in that it has a molecular weight from 2000 to 100 000 g/mol.
